(19) United States
(12) Patent Application Publication
(10) Pub. No.: US 2011/0218519 A1
(43) Pub. Date: Sep. 8, 2011

(54) DEVICE FOR INDICATING THE LIPOSUCTION QUANTITY AT THE SYMMETRIC PARTS OF HUMAN BODY AND LIPOSUCTION DEVICE

(75) Inventors: Yitao Zhou, Guangdong (CN); Pengwu Zhou, Guangdong (CN); Yahua Ttian, Guangdong (CN)

(73) Assignees: PENG'AI MEDICAL AESTHETIC HOSPITAL, Shenzhen, Guangdong (CN); SHENZHEN PENGCHENG HOSPITAL, Shenzhen, Guangdong (CN)

(21) Appl. No.: 13/126,967

(22) PCT Filed: Oct. 30, 2008
(86) PCT No.: PCT/CN2008/072878

§ 371 Date: Apr. 29, 2011

(51) Int. Cl. A61M 1/00

(52) U.S. Cl. 604/542

(57) ABSTRACT

A device for indicating the liposuction quantity at the symmetric parts of human body, which includes a buffer bottle (14) communicated with a suction pump (11) and a suction tube (17), and also includes a first liquid reserve bottle (15) and a second liquid reserve bottle (16), said first liquid reserve bottle (15) and said second liquid reserve bottle (16) are communicated with the buffer bottle (14) through tubes respectively, and said first liquid reserve bottle (15) and said second liquid reserve bottle (16) are connected to the soft suction tube (17) through tubes respectively.

### FIELD OF THE INVENTION

The present invention relates to medical plastic and cosmetic surgery field, and more particularly, to a device for indicating the liposuction quantity at the symmetric parts of human body and liposuction device.

### BACKGROUND OF THE INVENTION

Lipocyte of the human body increases along with the growth of the body in the growth and development stage. The lipocyte quantity keeps constant when grow up, which means when get fat after growing up, the lipocyte quantity never increases and only the volume of the lipocyte expands. When getting fat in the growth and development stage, the volume of the lipocyte expands and the lipocyte quantity increases at the same time.

When losing weight, whether for adult or minor, only the volume of the lipocyte reduces. Accordingly, when losing weight without surgery, the whole body weight is reduced, at the same time some parts of the body will appear to be corpulent and tumefacient comparing with the whole body due to the large quantity of lipocyte accumulated in the growth and development stage. At this time, the lipocyte accumulated at these parts only can be removed by surgery. Liposuction employs traditional surgical method to eliminate or adjust hypodermal lipocyte at different parts in the human body, so as to shape the human body and finish body lines.

It is well known that, symmetry is the principle of format beauty. The human body itself has unique symmetric beauty. The human body is a symmetric structure taking the midline of the body as the axis. For human, symmetry is not only exterior beauty but also requirement of health and survival. Symmetry is also a basic principle must be followed by a plastic and cosmetic surgery.

However, in the prior plastic and cosmetic surgery, one liquid reserve container is used to collect sucked liquid from different parts at the same time, to observe or record sucked liquid quantity of different parts in the surgery process. This manner is extremely likely to create observation or recordation error, which results in that lipocyte quantity sucked from each body may be different and then affects the symmetric effect and beauty of human body after the surgery.

### SUMMARY OF THE INVENTION

One aspect of the present invention is to provide a device for indicating the liposuction quantity at the symmetric parts of human body and liposuction device, so as to solving problem that the liposuction quantity at the symmetric parts of human body in liposuction surgery is hard to master in prior art.

The technical solution provided by present invention for solving above problem is providing a device for indicating the liposuction quantity at the symmetric parts of human body, which comprises a buffer bottle communicated with a suction pump and a suction soft tube, and also comprises a first liquid reserve bottle and a second liquid reserve bottle, the first liquid reserve bottle and the second liquid reserve bottle are communicated with the buffer bottle through a tube respectively, and the first liquid reserve bottle and the second liquid reserve bottle are communicated with the suction soft tube through a tube respectively.

In the device for indicating the liposuction quantity at the symmetric parts of human body according to present invention, the device further comprises a first tee, three ports of the first tee are communicated with the first liquid reserve bottle, the second liquid reserve bottle and the buffer bottle through a tube respectively.

In the device for indicating the liposuction quantity at the symmetric parts of human body according to present invention, the device further comprises a second tee, three ports of the second tee are communicated with the first liquid reserve bottle, the second liquid reserve bottle and the suction soft tube through a tube respectively.

In the device for indicating the liposuction quantity at the symmetric parts of human body according to present invention, the device further comprises a first valve body on the tube between the first tee and the first liquid reserve bottle, a second valve body on the tube between the first tee and the second liquid reserve bottle, a three valve body on the tube between the second tee and the first liquid reserve bottle, and a fourth valve body on the tube between the second tee and the second liquid reserve bottle.

In the device for indicating the liposuction quantity at the symmetric parts of human body according to present invention, the device further comprises a first switch device comprising two states, when the first switch device is in a first state, the second and fourth valve bodies are opened, and when the first switch device is in a second state, the second and fourth valve bodies are closed.

In the device for indicating the liposuction quantity at the symmetric parts of human body according to present invention, the device further comprises a second switch device comprising two states, when the second switch device is in a first state, the first and third valve bodies are opened, and when the second switch device is in a second state, the first and third valve bodies are closed.

In the device for indicating the liposuction quantity at the symmetric parts of human body according to present invention, the device further comprises a third switch device comprising two states, when the third switch device is in a first state, the first and third valve bodies are opened and the second and fourth valve bodies are closed, and when the third switch device is in a second state, the first and third valve bodies are closed and the second and fourth valve bodies are opened.

Another aspect of the present invention is to provide a liposuction device, which comprises a suction pump, a buffer bottle communicated with the suction pump, and a suction soft tube, and also comprises a first liquid reserve bottle and a second liquid reserve bottle, the first liquid reserve bottle and the second liquid reserve bottle are communicated with the buffer bottle through a tube respectively, and the first liquid reserve bottle and the second liquid reserve bottle are communicated with the suction soft tube through a tube respectively.

In the liposuction device according to present invention, the liposuction device further comprises a first tee and a second tee, three ports of the first tee are communicated with the first liquid reserve bottle, the second liquid reserve bottle and the buffer bottle through a tube respectively, three ports of the second tee are communicated with the first liquid reserve bottle, the second liquid reserve bottle and the suction soft tube through a tube respectively.

In the liposuction device according to present invention, the liposuction device further comprises a third switch device comprising two states, when the third switch device is in a first state, the first and third valve bodies are opened and the second and fourth valve bodies are closed, and when the third switch device is in a second state, the first and third valve bodies are closed and the second and fourth valve bodies are opened.

The device for indicating the liposuction quantity at the symmetric parts of human body and liposuction device of present invention, enable the liposuction quantity of the symmetric parts of human body is easier to control in liposuction surgery, by arranging two liquid reserve bottles for placing lipocyte from symmetric parts of human body. In such a way, effect and efficiency of the surgery can be improved.

### BRIEF DESCRIPTION OF THE DRAWINGS

Hereinafter, embodiments of present invention will be described in detail with reference to the accompanying drawings, wherein:

FIG. 1 is a diagram of an embodiment of a device for indicating the liposuction quantity at the symmetric parts of human body according to present invention;

FIG. 2 is a diagram showing liposuction by a first liquid reserve bottle;

FIG. 3 is a diagram showing liposuction by a second liquid reserve bottle.

### DETAILED DESCRIPTION OF THE PREFERRED EMBODIMENT

Referring to FIG. 1, is a structure diagram of an embodiment of a device for indicating the liposuction quantity at the symmetric parts of human body according to present invention. In present embodiment, the device of present invention comprises a buffer bottle 14 communicated with a suction pump 11 and a suction soft tube 17. The device also comprises first liquid reserve bottle 15, second liquid reserve bottle 16, first tee 12 and second tee 13. Wherein, three ports of the first tee 12 are communicated with the first liquid reserve bottle 15, the second liquid reserve bottle 16 and buffer bottle 14 through a tube respectively. Three ports of the second tee are 13 communicated with the first liquid reserve bottle 15, the second liquid reserve bottle 16 and suction soft tube 17 through a tube respectively. Through above manner, the first liquid reserve bottle 15 and the second liquid reserve bottle 16 are communicated with the buffer bottle 14 respectively, and the first liquid reserve bottle 15 and the second liquid reserve bottle 16 are communicated with the suction soft tube 17 respectively.

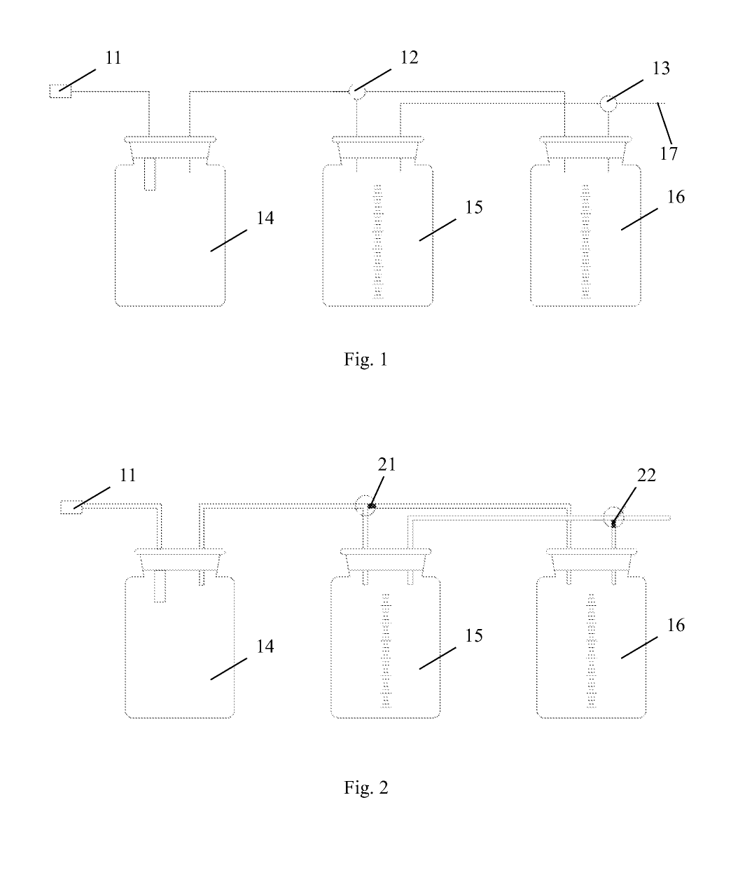

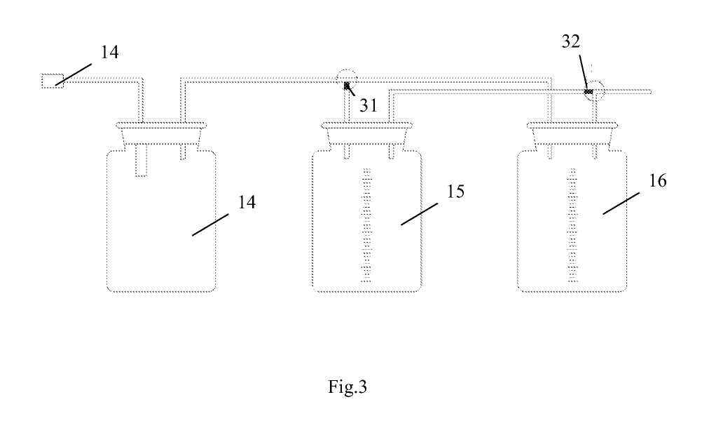

As shown in FIGS. 2, 3, the above device further comprises a first valve body 31 on the tube between the first tee 12 and the first liquid reserve bottle 15, a second valve body 21 on the tube between the first tee 12 and the second liquid reserve bottle 16, a three valve body 32 on the tube between the second tee 13 and the first liquid reserve bottle 15, and a fourth valve body 22 on the tube between the second tee 13 and the second liquid reserve bottle 16. Of course, the above valve bodies also can be combined with the tees, that is, a tee provided with a valve body is employed for controlling communication of the tube.

In the usage process of present invention, the opening and closing of the above mentioned four valve bodies can be adjusted in a manual manner. As shown in FIG. 2, the second and fourth valve bodies 21 and 22 are opened, the first and third valve bodies 31 and 32 are closed. The suction soft tube 17 is communicated with first liquid reserve bottle 15 via the second tee 13, and the first liquid reserve bottle 15 is communicated with the buffer bottle 14 via the first tee 12. In such a way, the suction soft tube 17 discharges the sucked lipocyte into the first liquid reserve bottle 15 under the function of the negative pressure generated by the suction pump 11. As shown in FIG. 3, when the second and fourth valve bodies 21 and 22 are closed, the first and third valve bodies 31 and 32 are opened, the suction soft tube 17 is communicated with second liquid reserve bottle 16 via the second tee 13, and the second liquid reserve bottle 16 is communicated with the buffer bottle 14 via the first tee 12. In such a way, the suction soft tube 17 discharges the sucked lipocyte into the second liquid reserve bottle 16 under the function of the negative pressure generated by the suction pump 11.

The first liquid reserve bottle 15 and second liquid reserve bottle 16 are used to reserve lipocyte of symmetric parts. Accordingly, the liposuction quantity can be handled easily, and unsymmetry after surgery can be avoided.

In above device, a first switch device (which isn't shown in the Figures, and can be such as an electromagnetic switch) can be used to control the opening and closing of the second and fourth valve bodies 21 and 22. The first switch device comprises two states, when the first switch device is in a first state, the second and fourth valve bodies 21 and 22 are opened, and when the first switch device is in a second state, the second and fourth valve bodies 22 and 24 are closed.

Similarly, in above device, a second switch device (which isn't shown in the Figures, and can be such as an electromagnetic switch) can be used to control the opening and closing of the first and third valve bodies 31 and 32. The second switch device comprises two states, when the second switch device is in a first state, the first and third valve bodies 31 and 32 are opened, and when the second switch device is in a second state, the second and fourth valve bodies 22 and 24 are closed.

In additional, in another embodiment, a third switch device (which isn't shown in the Figures, and can be such as a electromagnetic switch) can be used to control the opening and closing of the first valve body 31, the second valve body 32, the third valve body 31 and the fourth valve body 22. The third switch device comprises two states. When the third switch device is in a first state, the first and third valve bodies 31 and 32 are opened, and the second and fourth valve bodies 22 and 24 are closed. When the third switch device is in a second state, the first and third valve bodies 31 and 32 are closed, and the second and fourth valve bodies 22 and 24 are opened. Of course, the third switch device also can comprises a third state, that is, the above mentioned valve bodies are closed at the same time, so that, the device of present invention will stop work.

While the invention has been described with reference to certain aspects and embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A device for indicating the liposuction quantity at the symmetric parts of human body, which comprises a buffer bottle communicated with a suction pump and a suction soft tube, wherein the device also comprises a first liquid reserve bottle and a second liquid reserve bottle, the first liquid reserve bottle and the second liquid reserve bottle are communicated with the buffer bottle through a tube respectively, and the first liquid reserve bottle and the second liquid reserve bottle are communicated with the suction soft tube through a tube respectively.

2. The device for indicating the liposuction quantity at the symmetric parts of human body of claim 1, wherein the device further comprises a first tee, three ports of the first tee are communicated with the first liquid reserve bottle, the second liquid reserve bottle and the buffer bottle through a tube respectively.

3. The device for indicating the liposuction quantity at the symmetric parts of human body of claim 2, wherein the device further comprises a second tee, three ports of the second tee are communicated with the first liquid reserve bottle, the second liquid reserve bottle and the suction soft tube through a tube respectively.

4. The device for indicating the liposuction quantity at the symmetric parts of human body of claim 3, wherein the device further comprises a first valve body on the tube between the first tee and the first liquid reserve bottle, a second valve body on the tube between the first tee and the second liquid reserve bottle, a three valve body on the tube between the second tee and the first liquid reserve bottle, and a fourth valve body on the tube between the second tee and the second liquid reserve bottle.

5. The device for indicating the liposuction quantity at the symmetric parts of human body of claim 4, wherein the device further comprises a first switch device comprising two states, when the first switch device is in a first state, the second and fourth valve bodies are opened, and when the first switch device is in a second state, the second and fourth valve bodies are closed.

6. The device for indicating the liposuction quantity at the symmetric parts of human body of claim 4, wherein the device further comprises a second switch device comprising two states, when the second switch device is in a first state, the first and third valve bodies are opened, and when the second switch device is in a second state, the first and third valve bodies are closed.

7. The device for indicating the liposuction quantity at the symmetric parts of human body of claim 4, wherein the device further comprises a third switch device comprising two states, when the third switch device is in a first state, the first and third valve bodies are opened and the second and fourth valve bodies are closed, and when the third switch device is in a second state, the first and third valve bodies are closed and the second and fourth valve bodies are opened.

8. A liposuction device, which comprises a suction pump, a buffer bottle communicated with the suction pump, and a suction soft tube, wherein, the liposuction device also comprises a first liquid reserve bottle and a second liquid reserve bottle, the first liquid reserve bottle and the second liquid reserve bottle are communicated with the buffer bottle through a tube respectively, and the first liquid reserve bottle and the second liquid reserve bottle are communicated with the suction soft tube through a tube respectively.

9. The liposuction device of claim 8, wherein the liposuction device further comprises a first tee and a second tee, three ports of the first tee are communicated with the first liquid reserve bottle, the second liquid reserve bottle and the buffer bottle through a tube respectively, three ports of the second tee are communicated with the first liquid reserve bottle, the second liquid reserve bottle and the suction soft tube through a tube respectively.

10. The liposuction device of claim 11, wherein the liposuction device further comprises a third switch device comprising two states, when the third switch device is in a first state, the first and third valve bodies are opened and the second and fourth valve bodies are closed, and when the third switch device is in a second state, the first and third valve bodies are closed and the second and fourth valve bodies are opened.

11. The liposuction device of claim 9, wherein the liposuction device further comprises a first valve body on the tube between the first tee and the first liquid reserve bottle, a second valve body on the tube between the first tee and the second liquid reserve bottle, a three valve body on the tube between the second tee and the first liquid reserve bottle, and a fourth valve body on the tube between the second tee and the second liquid reserve bottle.

12. The liposuction device of claim 11, wherein the liposuction device further comprises a first switch device comprising two states, when the first switch device is in a first state, the second and fourth valve bodies are opened, and when the first switch device is in a second state, the second and fourth valve bodies are closed.

13. The liposuction device of claim 11, wherein the liposuction device further comprises a second switch device comprising two states, when the second switch device is in a first state, the first and third valve bodies are opened, and when the second switch device is in a second state, the first and third valve bodies are closed.
